(19) United States
(12) Patent Application Publication
(10) Pub. No.: US 2025/0378350 A1
(43) Pub. Date: Dec. 11, 2025

(54) METHOD AND SYSTEM FOR DETERMINING UNCERTAINTY IN PERSONALIZED FEDERATED LEARNING

(71) Applicant: Mohamed bin Zayed University of Artificial Intelligence, Abu Dhabi (AE)

(72) Inventors: Maxim PANOV, Abu Dhabi (AE); Nikita KOTELEVSKII, Abu Dhabi (AE); Martin TAKAC, Abu Dhabi (AE); Samuel HORVATH, Abu Dhabi (AE)

(73) Assignee: Mohamed bin Zayed University of Artificial Intelligence, Abu Dhabi (AE)

(21) Appl. No.: 18/738,622

(22) Filed: Jun. 10, 2024

(51) Int. Cl. G06N 3/098; G16H 50/20

(52) U.S. Cl. CPC G06N 3/098; G16H 50/20

(57) ABSTRACT

A method and system for uncertainty quantification approach for federated learning that enables the distinction between aleatoric and epistemic uncertainties, as well as between local and global in-and out-of-distribution data. The method and system offer permit selecting the appropriate model to predict on a given input based on these uncertainty estimations. This comprehensive framework contributes to enhancing the robustness and reliability of federated learning models in real-world applications, effectively addressing the challenges that arise due to the heterogeneity and diverse nature of data distributions.

### STATEMENT REGARDING PRIOR DISCLOSURE BY THE INVENTORS

Aspects of this technology are described in an article Kotelevskii, Nikita, Samuel Horváth, Karthik Nandakumar, Martin Takáč, and Maxim Panov. “Dirichlet-based Uncertainty Quantification for Personalized Federated Learning with Improved Posterior Networks.” arXiv preprint arXiv: 2312.11230 (2023) and is herein incorporated by reference in its entirety.

### BACKGROUND

### Technical Field

The present disclosure relates to artificial intelligence and particularly to an uncertainty quantification approach for federated learning to distinguish between aleatoric and epistemic uncertainties, and local and global in-and out-of-distribution data.

### Description of the Related Art

The widespread adoption of deep neural networks in various applications requires reliable predictions, which can be achieved through rigorous uncertainty quantification. Although uncertainty quantification has been extensively studied in different domains under centralized settings, only a few works have considered this area within the context of federated learning. See Kimin Lee, Kibok Lee, Honglak Lee, and Jinwoo Shin. A simple unified framework for detecting out-of-distribution samples and adversarial attacks. In S. Bengio, H. Wallach, H. Larochelle, K. Grauman, N. Cesa-Bianchi, and R. Garnett, editors, Advances in Neural Information Processing Systems, volume 31. Curran Associates, Inc., 2018; Balaji Lakshminarayanan, Alexander Pritzel, and Charles Blundell. Simple and scalable predictive uncertainty estimation using deep ensembles. Advances in neural information processing systems, 30, 2017; Yarin Gal and Zoubin Ghahramani. Dropout as a bayesian approximation: Representing model uncertainty in deep learning. In International Conference on Machine Learning, pages 364 1050-1059. PMLR, 2016; Nikita Kotelevskii, 387 Aleksandr Artemenkov, Kirill Fedyanin, Fedor Noskov, Alexander Fishkov, Artem Shelmanov, Artem Vazhentsev, Aleksandr Petiushko, and Maxim Panov. Nonparametric uncertainty quantification for single deterministic neural network. Advances in Neural Information Processing Systems, 35:36308-36323, 2022; Nikita Kotelevskii, Maxime Vono, Alain Durmus, and Eric Moulines. Fedpop: A bayesian approach for personalised federated learning. Advances in Neural Information Processing Systems, 35:8687-8701, 2022; and Florian Linsner, Linara Adilova, Sina Daubener, Michael Kamp, and Asja Fischer. Approaches to uncertainty quantification in federated deep learning. In Machine Learning and Principles and Practice of Knowledge Discovery in Databases: International Workshops of ECML PKDD 2021, Virtual Event, Sep. 13-17, 2021, Proceedings, Part I, pages 128-145. Springer, 2022. Typically, in federated learning papers, algorithms result in using either a personalized local model or a global model. However, both these models could be useful in different cases by providing the tradeoff between personalization of a local model and higher reliability of the global one. See Filip Hanzely and Peter Richtarik. Federated learning of a mixture of global and local models. arXiv preprint arXiv: 2002.05516, 2020; and Paul Pu Liang, Terrance Liu, Liu Ziyin, Nicholas B Allen, Randy P Auerbach, David Brent, Ruslan Salakhutdinov, and Louis-Philippe Morency. Think locally, act globally: Federated learning with local and global representations. arXiv preprint arXiv: 2001.01523, 2020.

An ensemble of K global models as the best approach for federated uncertainty quantification, which is K times more expensive compared to the classical FedAvg method has been proposed. A Markov Chain Monte Carlo (MCMC) to obtain samples from a posterior distribution, which is practically almost infeasible due to its computational complexity, has been proposed.

Other work could potentially be utilized for estimating uncertainty in federated learning. See Hong-You Chen and Wei-Lun Chao. Fedbe: Making Bayesian model ensemble applicable to federated learning. In ICLR, 2021; and Minyoung Kim and Timothy Hospedales. Fedhb: Hierarchical bayesian federated learning. arXiv preprint arXiv:2305.04979, 2023. However, these studies do not explicitly discuss the opportunities and challenges associated with uncertainty quantification in their papers.

Posterior Networks (PostNet) and its modification, Natural Posterior Networks (NatPN) involve using a Dirichlet prior and posterior distributions over categorical predictive distributions. See Bertrand Charpentier, Daniel Zugner, and Stephan Gunnemann. Posterior network: Uncertainty estimation without good samples via density-based pseudo-counts. Advances in Neural Information Processing Systems, 33:1356-1367, 2020; and Bertrand Charpentier, Oliver Borchert, Daniel Zugner, Simon Geisler, and Stephan Gunnemann. Natural posterior network: Deep Bayesian predictive uncertainty for exponential family distributions. In International Conference on Learning Representations, 2022, each incorporated herein by reference in their entirety.

To parameterize the parameters of these Dirichlet distributions, the use of a density model over the deep representations of input objects has been proposed. See Andrey Malinin and Mark Gales. Predictive uncertainty estimation via prior networks. Advances in neural information processing systems, 31, 2018; Andrey Malinin and Mark Gales. Reverse kl-divergence training of prior networks: Improved uncertainty and adversarial robustness. Advances in Neural Information Processing Systems, 422 32, 2019; and Murat Sensoy, Lance Kaplan, and Melih Kandemir. Evidential deep learning to quantify classification uncertainty. Advances in neural information processing systems, 31, 2018, each incorporated herein by reference in their entirety. In NatPN, a Normalizing Flow is employed to estimate the density of embeddings extracted by a trained feature extractor. See George Papamakarios, Eric T Nalisnick, Danilo Jimenez Rezende, Shakir Mohamed, and Balaji Lakshminarayanan. Normalizing flows for probabilistic modeling and inference. J. Mach. Learn. Res., 22(57):1-64, 2021; and Ivan Kobyzev, Simon JD Prince, and Marcus A Brubaker. Normalizing flows: An introduction and review of current methods. IEEE transactions on pattern analysis and machine intelligence, 43(11):3964-3979, 2020. This density is then used to calculate updates to the Dirichlet distribution.

Despite the success of the NatPN model, there are certain issues with the loss function employed in NatPN, which become particularly critical when dealing with high aleatoric regions (which could be a potential issue in federated learning). Other potential issues related to the training of Dirichlet models in general are known but there are no solutions to address these challenges. See Viktor Bengs, Eyke Hullermeier, and Willem Waegeman. Pitfalls of epistemic uncertainty quantification through loss minimisation. In Advances in Neural Information Processing Systems, 2022; and Viktor Bengs, Eyke Hullermeier, and Willem Waegeman. On second-order scoring rules for epistemic uncertainty quantification. arXiv preprint arXiv:2301.12736, 2023, each incorporated herein by reference in their entirety.

Federated learning systems are exposed to challenges due to the inherent heterogeneity and diverse nature of data distributions across different nodes, which can compromise the robustness and reliability of models.

The task of accurately quantifying uncertainty in such models is a significant issue that has not been adequately addressed. Current approaches struggle with differentiating between aleatoric and epistemic uncertainties, and also distinguishing between local and global in-and out-of-distribution data. This differentiation is vital for improving the model's performance and making reliable predictions in a federated learning environment. Additionally, there is a need for a method that uses these uncertainty estimations to select the appropriate model to predict on a given input. These issues form the basis of the problem addressed herein and provide a new uncertainty quantification approach for federated learning that enhances model robustness and reliability.

Accordingly, it is one object of the present disclosure to provide methods and systems for a framework to choose whether to predict with a local or global model at a given point based on uncertainty quantification. An object is to apply the global model only if the local one has high epistemic (model) uncertainty about the prediction at a given point, i.e., the local model doesn't have enough information about the particular input point. An object includes in case the local model is confident (either in predicting a particular class or in the fact that it is observing an ambiguous object with high aleatoric (data uncertainty), it should make the decision itself without involving the global one.

### SUMMARY OF THE INVENTION

In one aspect the present disclosure includes a federated learning system, including a local neural network model; a global neural network model in communication with each of the local neural network models, a selector configured to switch between use of the global model for prediction only if the local model has high epistemic (model) uncertainty about the prediction at a given point, in which the local model makes unreliable predictions at this particular input point. When the local model is confident (either in predicting a particular class or in the fact that it is observing an ambiguous object with high aleatoric (data) uncertainty), make the decision by the local model without involving the global one.

In another aspect, the present disclosure includes a local model and a global model each which determines aleatoric uncertainty in which if the aleatoric uncertainty is above a threshold both the local model and the global model abstain from prediction.

In another aspect, the present disclosure includes a training loss function that simultaneously maximizes likelihood of embeddings and prevents potential impact of a Bayesian loss on density estimation parameters.

In another aspect, the present disclosure includes choosing the threshold chosen based on an additional calibration dataset.

In another aspect, the present disclosure includes a hospital model that is trained from local hospital patient data, and in which when the local hospital patient data does not fit the local statistical distribution, the selector of a respective workstation switches to downloading trained global weights of the global model to the local model.

In another aspect, the present disclosure includes a medical diagnosis system in a network, the system can include a plurality of workstations for a plurality of respective medical facilities, where each workstation performs medical diagnosis using medical data that is unique to the respective medical facility; a central server, connected to communicate with the plurality of workstations, for maintaining a global model; wherein each of the workstations maintains a local model for the medical diagnosis, wherein each of the workstations includes a selector configured to switch between: (i) use of the global model for the medical diagnosis only if the local model has high epistemic uncertainty about the diagnosis at a given input point, wherein the local model has high epistemic uncertainty based on a quantity of data about a particular input point that is less than a predetermined uncertainty threshold, (ii) use of the local model for the medical diagnosis when the local model is confident, either in predicting a particular medical diagnosis or when predicting an ambiguous diagnosis with aleatoric uncertainty that is above the predetermined uncertainty threshold.

In another aspect, the present disclosure includes a method of medical diagnosis in a network including a plurality of workstations for a plurality of respective medical facilities, where each workstation maintains a local model for medical diagnosis, and a central server, connected to communicate with the plurality of workstations, for maintaining a global model for the medical diagnosis, the method can include performing, in the plurality of workstations, the medical diagnosis using medical data that is unique to the respective medical facility; switching, in each of the workstations, between (i) use of the global model for the medical diagnosis only if the local model has high epistemic uncertainty about the diagnosis at a given input point, wherein the local model has high epistemic uncertainty above a predetermined uncertainty threshold that is based on a quantity of data about a particular input point that is less than a predetermined quantity, (ii) use of the local model for the medical diagnosis when the local model is confident, either in predicting a particular medical diagnosis or when predicting an ambiguous diagnosis with aleatoric uncertainty that is above the predetermined uncertainty threshold.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a non-limiting network with machine learning models trained through federated learning;

FIG. 2 illustrates a display screen in a client of the network;

FIG. 3 shows a general distributed scheme where each input is first processed by personalized local model, according to an exemplary aspect of the disclosure;

FIG. 4 is a block diagram of a machine learning model, according to an exemplary aspect of the disclosure;

FIG. 5 is a flow diagram of a natural posterior network, as an exemplary implementation of the machine learning model;

FIG. 6A is a graph of a landscape of the loss function with K=10 classes, showing a. line, representing high aleatoric regions.

FIG. 6B is a plot of training data, where the leftmost and rightmost Gaussians consist of only one class each;

FIG. 6C is a graph that shows that by altering the number of classes K, the density of extracted representations, learned by a normalizing flow, does not vanish for the loss function.

FIG. 7 is a flow diagram of an encoder for the network of FIG. 5;

FIG. 8 is a graph of the variation in precision as a function of the filtering threshold;

FIG. 9 is an algorithm for training a machine learning model, according to an exemplary aspect of the disclosure;

FIG. 10A is a histogram depicting aleatoric uncertainty, represented as the expected entropy of the predictive distribution;

FIG. 10B is a histogram of logarithms of density, assigned to noisy (high aleatoric) and clean (low aleatoric) objects.

FIGS. 11A-11G display matrices of accuracy for varying models, each assessed across distinct classes;

FIG. 12 is a block diagram of a computer system for implementing federated learning of the present disclosure.

### DETAILED DESCRIPTION OF THE INVENTION

In the drawings, like reference numerals designate identical or corresponding parts throughout the several views. Further, as used herein, the words “a,” “an” and the like generally carry a meaning of “one or more,” unless stated otherwise.

The present disclosure addresses the problem of uncertainty quantification in federated learning. Aspects of this disclosure are directed to a system, framework, and method of federated learning based on uncertainty quantification, which allows switching between using a local or global model. A federated learning framework uses Local-Confident, Local-Ambiguous, Local-OOD, and Global-Uncertain to enable us to reason about the choice of model for prediction. An aspect of the framework, referred to as FedPN, uses the Dirichlet-based NatPN model. For this particular model, an aspect is a solution to an issue in the loss function of NatPN (not known in literature before) that complicates disentanglement of aleatoric and epistemic uncertainties.

An embodiment of the framework is based on the Posterior Networks (PostNet) and its modification, Natural Posterior Networks (NatPN). This type of model is particularly useful, as it enables the estimation of aleatoric and epistemic uncertainties without incurring additional inference costs. Thus, the switching between local and global models is implemented in an efficient way.

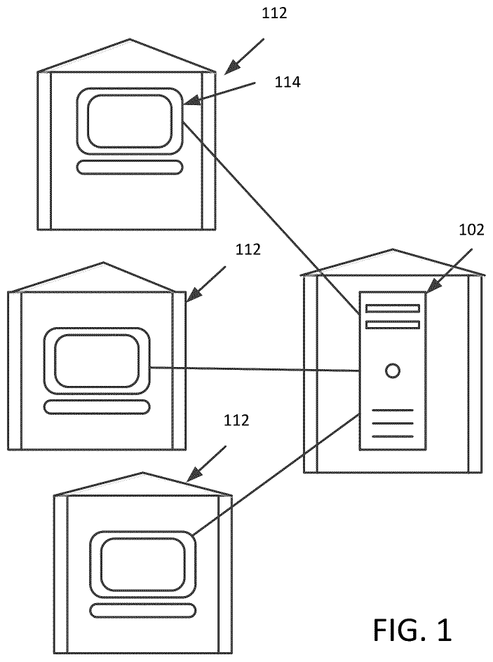

FIG. 1 is a non-limiting network with machine learning models trained through federated learning. In one embodiment, multiple hospitals 112 come together to create a machine learning model for medical diagnosis using their unique patient data. Each hospital includes at least one client workstation 114, where the client workstations 114 are connected to a server 102 that maintains a global machine learning model. Each hospital 112 maintains its own local patient data, including medical images.

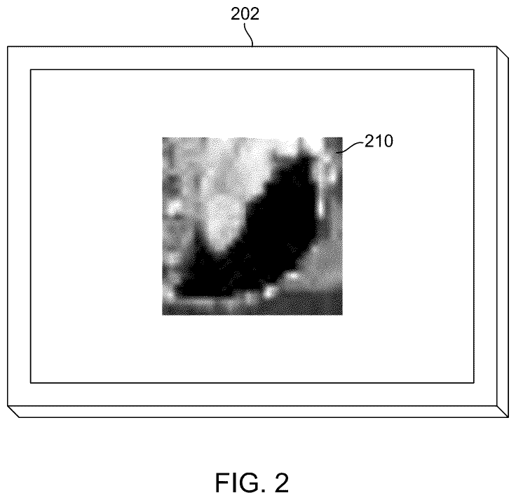

FIG. 2 illustrates a display screen in a client workstation of the network. A client workstation 114 can display medical images 210 in a display screen 202. A client workstation 114 can be configured with a local machine learning model for medical diagnosis using the medical image 210.

Federated Learning is used for developing a global machine learning model, but concurrently, each hospital formulates its own machine learning model strictly based on its local data, giving two different-models: local and global ones. Should a local model produce reliable predictions, there is no need to depend on the global machine learning model, which might not perform as well due to the diversity between hospital's data. However, if a patient's data does not fit the local data distribution patterns, this strategy can identify this anomaly and advise the usage of the global model. The global model may offer better insights as it's trained on a broader and more diverse set of data.

### General Framework of Switching Between Global and Personal Models

A description of the general idea and potential nuances is provided first. A description of a specific implementation is provided later.

### A. Concept Overview

Federated learning involves multiple clients, each having its own personalized local model. However, in the present disclosure, it is assumed that a global model is available. The global model is typically expected to perform reasonably well on each client's data. Assuming that there are trained global and local models, there may be a situation where clients have the option to use either the global model or their local model to make predictions for a new unseen object image x.

The choice between local and global models for prediction depends on the multiple factors that contribute to their prediction quality. First of all, shifts in the distribution between the local data of a particular client and the global population may have a significant effect on the models' performance. Possible shifts include covariate shift, label shift or different types of label noise. If the shift is significant, the global model might be very biased with respect to the prediction for the particular client, while normally the local model is unbiased. The second part of the picture is the size of the available data. Generally, the global model has more data to work with and potentially, if no data shift is present, should outperform the local one. However, the global model is usually trained with no direct access to the data stored at clients, which might degrade its performance. Eventually, the best performing model will be the one which achieves better bias-variance trade-off.

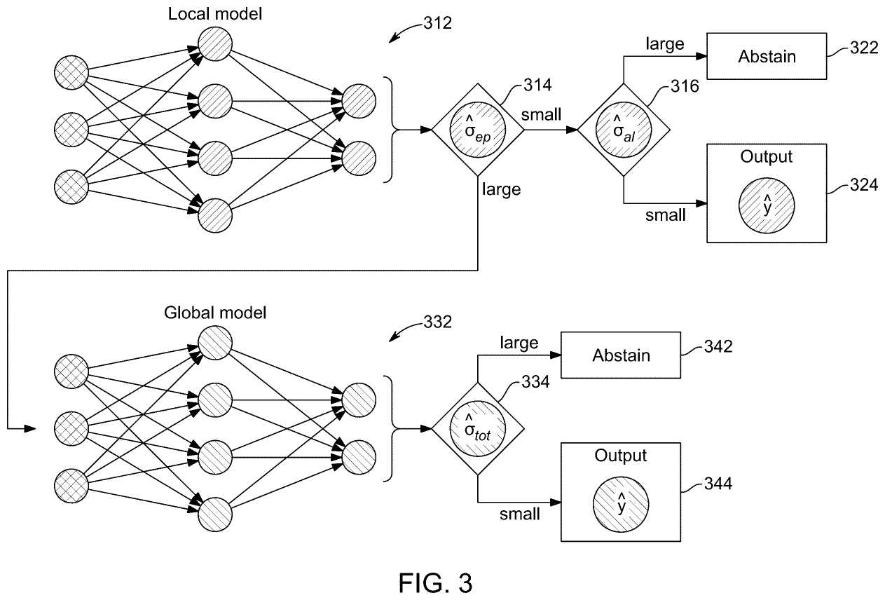

The disclosed framework chooses between pointwise usage of local or global models for prediction based on the uncertainty scores provided by the model (or abstain from making a prediction altogether, explicitly acknowledging the uncertainty). Both local and global models can provide uncertainty estimates corresponding not only to the total predictive uncertainty but also to separate aleatoric and epistemic uncertainties. The workflow for the framework is summarized in FIG. 3.

Regarding FIG. 3, each input is first processed by personalized local model 312. In case of high epistemic uncertainty (large in 314), the decision is delegated to the global model 332, which can output a prediction 344. Otherwise (small in 314), if epistemic uncertainty is low (small in 316), local model 312 proceeds with the decision 324. Both models also consider aleatoric uncertainty 316, 334 and may abstain from prediction, 322, 342.

### B. Choice of Model

An important fact is that the local model is not exposed to the data shift between the general population and the particular client. Thus, if this local model is sufficiently confident in the prediction, there is no need to involve the global model at all. However, it is important to distinguish between different types of uncertainty. Usually, the total uncertainty of the model predicting at a particular data point can be split into two parts: aleatoric uncertainty and epistemic uncertainty. Aleatoric uncertainty is the one that reflects the inherent noise in the data. The epistemic uncertainty is the one that reflects the lack of knowledge due to the fact that the model was trained on a data set of a limited size.

In one embodiment, it is extremely important to distinguish between aleatoric and epistemic uncertainties. Referring back to FIG. 3, suppose the local model 312 has low epistemic (small in 314) and high aleatoric uncertainty (large in 316) at some point. In that case, the model is confident that the predicted label is ambiguous, and the model should abstain 322 from prediction. However, if the epistemic uncertainty is high (large in 314), it means that the local model 312 does not have enough knowledge to make the prediction (enougi.e., the quantity of data is below a predetermined quantity threshold), and the global model 332 should make the decision. For purposes of this disclosure, the predetermined quantity threshold is determined based on the type of data. In the case of medical image data, the quantity of local image data is considered insufficient in the field for rare diseases. The quantity of such rare disease image data may become sufficient in a medical facility that specializes in treatment for such a rare disease. In other words, the quantity threshold may be set such that a percentage of total local images that show the subject rare disease is sufficient. The quantity threshold may be set such that over time a quantity of image data becomes sufficient for the particular rare disease. As an example, a quantity threshold is set when at least 3% of the total image data includes the subject disease and there are at least 100 total images. The global model 332, in its turn, may either proceed with the prediction 344 when it is confident or abstain 342 from prediction when there is high uncertainty (large in 334) associated with the prediction. Thus, in this context, for a fixed client and an unseen input, there are four possible outcomes, see Table 1.

TABLE 1
Possible scenarios for the input data point in introduced setup. A particular
input falls in one of the categories depending on the confidence in its prediction
by local and global models. The disentanglement between alcalorie and epistemic
uncertainties is crucial to make the decision in an optimal way.

| | |
|---|---|
| Known knowns | Known unknowns |
| Local Confident. This represents local | Local Ambiguous. This is local in- |
| in-distribution data for which the local | distribution data with high aleatorie |
| model is confident in prediction. | uncertainty (class ambiguity). |
| Unknown knowns | Unknown unknowns |
| Local OOD. This refers to data that is | Global Uncertain. These input data is |
| locally unknown (high epistemic uncertainty) | out-of-distribution for the local model |
| but known to other clients. In this case, | while the global model is uncertain in |
| it makes sense to use the global model for | prediction (high total uncertainty). The |
| predictions. | best course of action is to abstain from |
| | making a prediction. |

The particular implementation of the approach described above depends on the choice of the machine learning model and the way to compute uncertainty estimates. The key feature required is the ability of the method to compute both aleatoric and epistemic uncertainties. The implementation of the present method and system is based on the posterior networks framework.

### Dirichlet-Based Deep Learning Models

The Dirichlet-based models are a specific instance of the general framework. The intuition behind this decision lies in the fact that these models allow the distinction between various types of uncertainty and facilitate the computation of corresponding uncertainty estimates with minimal additional computational overhead. Furthermore, unlike ensemble methods, there is no need to train multiple models. See William H Beluch, Tim Genewein, Andreas Nurnberger, and Jan M Kohler. The power of ensembles for active learning in image classification. In Proceedings of the IEEE conference on computer vision and pattern recognition, pages 9368-9377, 2018, incorporated herein by reference in its entirety. In comparison to approximate Bayesian techniques, such as MC Dropout or Variational Inference, almost all expectations of interest can be derived in closed form. See Gal et al.; and Alex Graves. Practical variational inference for neural networks. Advances in neural information processing systems, 24, 2011, incorporated herein by reference in their entirety. This makes Dirichlet-based models an attractive and efficient option for implementing the present framework.

### A. Basics of Dirichlet-Based Models

The basics of Dirichlet-based models are first provided for classification tasks. To ease the introduction, start by considering a training dataset

$$D = {\{ {x}_{i} , {y}_{i} \}}_{i = 1}^{N} ,$$

where N denotes the total number of data points in the dataset. It is assumed that labels yᵢ belong to one of K classes.

Typically, the Dirichlet-based approaches assume that the model consists of two hierarchical random variables, μ and θ. The posterior predictive distribution for a given unseen object x can be computed as follows:

$$p ( y \vert x , D ) = \int p ( y \vert μ ) [ \int p ( μ \vert x , θ ) p ( θ \vert D ) d θ ] d μ ,$$

where p(y|μ) is the distribution over class labels, given some probability vector (e.g., Categorical), p(μ|x, θ) is the distribution over a simplex (e.g., Dirichlet), and p(θ|D) is the posterior distribution over parameters over the model.

However, for practical neural networks, the posterior distribution p(θ|D) does not have an analytical form and is computationally intractable. The “semi-Bayesian” scenario is used by looking on a point estimate of this distribution: p(D)=δ(θ−θ\hat{ }), where θ0\hat{ } is some estimate of the parameters (e.g., MAP estimate). Then the integral inside the brackets simplifies:

$$\int p ( μ \vert x , θ ) p ( θ \vert D ) d θ = \int p ( μ \vert x , θ ) δ ( θ - \hat{θ} ) d θ = p ( μ \vert x , \hat{θ} ) .$$

In the series of works the posterior distribution p(μ|x, θ\hat{ }) is chosen to be the Dirichlet distribution Dir(μ|α^{post}(x)) with the parameter vector α^{post}(x)=α^{post}(x|θ\hat{ }) that depends on the input point x. In these models, the prior over probability vectors μ takes the form of a Dirichlet distribution, representing the distribution over beliefs about the probability of each class label. In other words, it is a distribution over distributions of class labels. This prior is parameterized by a parameter vector α^{prior}, where each component

$${α}_{c}^{prior}$$

corresponds to a belief in a specific class. PostNet and NatPN propose the idea that the posterior parameters α^{post}(x) can be computed in the form of pseudo-counts, parameterized by a function:

$${α}^{post} ( x ) = {α}^{prior} + α ( x ) , \qquad ( 1 )$$

where α(x)=α(x|θ\hat{ }) is a function of input object x that maps it to positive values. Parameterization of α(x). In NatPN it is proposed to use the following parameterization:

$$α ( x ) = p ( g ( x ) ) f ( g ( x ) ) . \qquad ( 2 )$$

In this parameterization, g(x) represents a feature extraction function that maps the input object x (usually high-dimensional) to a lower-dimensional embedding. Subsequently, p(⋅) is a “density” function (parameterized by normalizing flow), and f(⋅) is a function mapping the extracted features to a vector of class probabilities.

This parameterization offers several advantages. Firstly, since p(⋅) is expected to represent the density of training examples, it should be high for in-distribution data. Secondly, as the density is properly normalized, embeddings that lie far from the training ones will result in lower values of p(g(x)), thus leading to lower α(x). This means that for such input x, any evidence will not be added, and consequently, α^{post}(x) will be close to α^{prior}.

### B. Uncertainty Measures for Dirichlet-Based Models

One of the advantages of using Dirichlet-based models is their ability to easily disentangle and quantitatively estimate aleatoric and epistemic uncertainties.

### Epistemic Uncertainty.

First epistemic uncertainty is discussed, which can be estimated in multiple different ways. See Andrey Malinin and Mark Gales. Uncertainty estimation in autoregressive structured prediction. In International Conference on Learning Representations, 2021, incorporated herein by reference in its entirety. In the present disclosure the epistemic uncertainty is quantified as the entropy of a posterior Dirichlet distribution, which can be analytically computed as follows

$$ℋ [ Dir ( μ \vert {α}^{post} ( x ) ) ] = ln \frac{{\prod }_{i = 1}^{K} Γ ( {α}_{i}^{post} ( x ) )}{Γ ( {α}_{0}^{post} ( x ) )} - {\sum }_{i = 1}^{K} ( {α}_{i}^{post} ( x ) - 1 ) ( ψ ( {α}_{i}^{post} ( x ) ) - ψ ( {α}_{0}^{post} ( x ) ) ) , \qquad ( 3 )$$

### Aleatoric Uncertainty.

Aleatoric uncertainty can be measured using the average entropy, which can be computed as follows:

$${𝔼}_{μ \sim Dir ( μ \vert {α}^{post} ( x ) )} ℋ [ p ( y \vert μ ) ] = - {\sum }_{i = 1}^{K} \frac{{α}_{i}^{post} ( x )}{{α}_{0}^{post} ( x )} [ ψ ( {α}_{i}^{post} ( x ) + 1 ) - ψ ( {α}_{0}^{post} ( x ) + 1 ) ] . \qquad ( 4 )$$

See Alex Kendall and Yarin Gal. What uncertainties do we need in bayesian deep learning for computer vision? Advances in neural information processing systems, 30, 2017, incorporated herein by reference in its entirety.

This metric captures the inherent noise present in the data, thus providing an estimate of the aleatoric uncertainty.

In the next section it is shown how Dirichlet-based models and uncertainty measures based on them can be used in federated learning.

### FedPN: NatPN Goes Federated

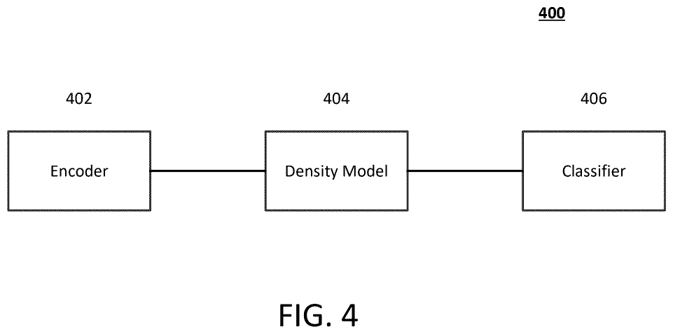

FIG. 4 is a block diagram of a machine learning model. The machine learning model 400 is of the same architecture for each local machine learning model and the global machine learning model. The model 400 includes an encoder 402 for mapping an input image to a low-dimensional latent representation, a density model (normalized flow 404) for updating parameters, and a classifier 406 for determining a predicted class.

Client workstations that maintain local machine learning models are given an array of datasets Dᵢ for 1≤i≤b, where b represents the number of clients. Each sei or data

$${D}_{i} = {\{ {x}_{j}^{i} ∈ {R}^{d} , {y}_{j}^{i} \}}_{j = 1}^{\vert {D}_{i} \vert}$$

consists of object-label pairs. The federated framework is configured in such a way that all clients share the feature extractor g parameters ϕ (in encoder 402) and maintain personalized heads fⁱ parameterized by θᵢ (in classifier 406). Furthermore, a “global” head-model f (classifier 406) is trained using the FedAvg (and SCAFFOLD) method and ultimately has parameters θ. See Brendan McMahan, Eider Moore, Daniel Ramage, Seth Hampson, and Blaise Aguera y Arcas. Communication-efficient learning of deep networks from decentralized data. In Artificial intelligence and statistics, pages 1273-1282. PMLR, 2017; and Sai Praneeth Karimireddy, Satyen Kale, Mehryar Mohri, Sashank Reddi, Sebastian Stich, and Ananda Theertha Suresh. Scaffold: Stochastic controlled averaging for federated learning. In International Conference on Machine Learning, pages 5132-5143. PMLR, 2020, each incorporated herein by reference in their entirety.

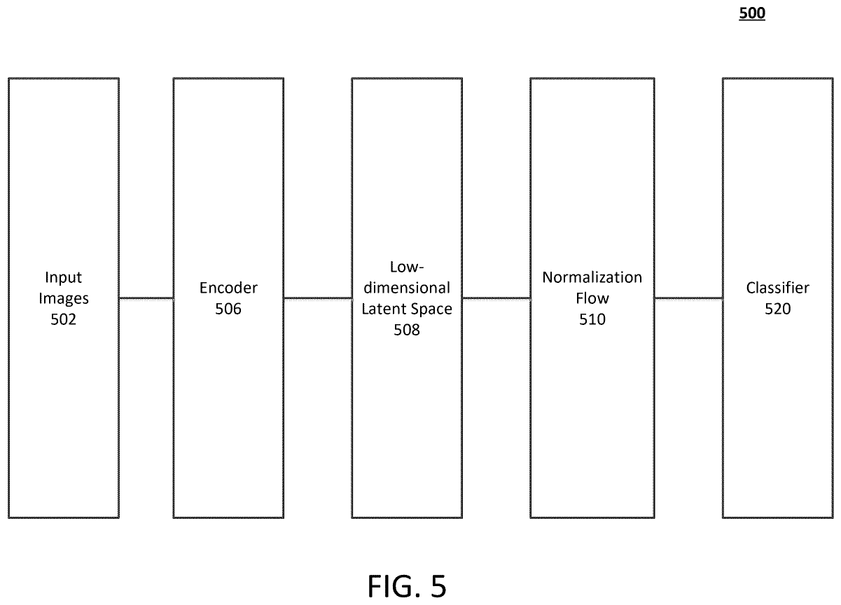

Following the NatPN approach, normalizing flows are employed to estimate the embedding density. FIG. 5 is a flow diagram of a natural posterior network (NatPN), as an exemplary implementation of the machine learning model.

Input images x⁽ⁱ⁾ 502 are first mapped to a low-dimensional latent representation z⁽ⁱ⁾ 508 by the encoder f_{ϕ} 506. From z⁽ⁱ⁾, the normalizing flow P_̅{̅ω̅}̅ 510 yields the evidence update n⁽ⁱ⁾. Posterior parameters X^{post} and n^{post} are obtained from a weighted combination of prior and update parameters according to n^{post;(i)}.

In general, the normalizing flow 510 describes the transformation of a probability density through a sequence of invertible mappings. By repeatedly applying the rule for change of variables, the initial density flows through the sequence of invertible mappings. A normalized flow that applies radial contractions and expansions around the reference point are thus referred to as radial flows.

As it is for head models, two types of models—local density models pⁱ (using local data) parameterized by ψᵢ and global density model p (using data from other clients in a federated fashion) parameterized by ψ are learned. It is important to note that both types of models are trained on the same domain since the feature extractor model (encoder 402) is fixed for local and global models.

The loss function is the expected cross-entropy, also known as Uncertain Cross Entropy (UCE). See Marin Biloš, Bertrand Charpentier, and Stephan Gunnemann. Uncertainty on asynchronous time event prediction. Advances in Neural Information Processing Systems, 32, 2019, incorporated herein by reference in its entirety. Note, that this loss function is a strictly proper scoring rule, which implies that a learner is incentivized to learn the true conditional p(y|x). For a given input x, the loss can be written as:

$$L ( y , {α}^{post} ( x ) ) = {𝔼}_{μ \sim Dir ( μ \vert {α}^{post} ( x ) )} {\sum }_{i = 1}^{K} - 𝟙 [ y = i ] log {μ}_{i} d μ = ψ ( {α}_{0}^{post} ( x ) ) - ψ ( {α}_{y}^{post} ( x ) ) , \qquad ( 5 )$$

where

$${α}_{0}^{p o s t} ( x ) = {\sum }_{i = 1}^{K} {α}_{i}^{p o s t} ( x ) .$$

Additionally, authors suggest to penalize too concentrated predictions, by adding the regularization term with some hyperparameter λ. The overall loss function looks as follows:

$$L ( y , {α}^{post} ( x ) ) - λℋ [ Dir ( μ \vert {α}^{post} ( x ) ) ] , \qquad ( 6 )$$

where H denotes the entropy of a distribution. This overall loss function referred by authors as Bayesian loss.

Issues with loss functions. An asymptotic form of (5) is formed. For all x>0, the following inequality holds:

$$log x - \frac{1}{x} \leq ψ ( x ) \leq log x - \frac{i}{2 x} .$$

Recalling the update rule (1) and using the specific parameterization of

$${α}_{c}^{prior} = 1$$

for all c, we conclude that all

$${α}_{c}^{post} ( x ) > 1 .$$

Hence, we can approximate

$$ψ ( {α}_{c}^{post} ( x ) ) \approx log {α}_{c}^{post} ( x ) .$$

To simplify the notation, use z=g(x):

$$L ( y , {α}^{post} ( x ) ) \approx log ( {α}_{0}^{post} ( x ) ) - log ( {α}_{y}^{post} ( x ) ) = log K + log [ 1 + \frac{p ( z ) ( \frac{1}{K} - {f}_{y} ( z ) )}{{α}_{y}^{prior} + p ( z ) {f}_{y} ( z )} ] , \qquad ( 7 )$$

see the full derivation below.

It can be observed from (7) that for an in-distribution case with high aleatoric uncertainty (when all classes are confused and equally probable), the last term is canceled. Note, that the presence of entropy term resulting to the final loss of equation (6)), which incentives learner to produce smooth prediction will only amplify the effect. This implies that no gradients concerning the parameters of the density model will be propagated. As a result, p(⋅) may disregard regions in the embedding space that correspond to areas with a high concentration of ambiguous training examples. This violates the intuition of p(⋅) as a data density. Thus, uncertainty estimates based on p(⋅) cannot be used to measure epistemic uncertainty, as it ignores the regions with high aleatoric uncertainty.

In addition to the problem of confusing high-aleatoric and high-epistemic regions, it is discovered that the loss function defies intuition when confronted with “outliers.” An “outlier” is defined as an object with a predicted probability less than 1/K. From (7), it can be observed that the last term changes its sign precisely at the point where

$${f}_{y} ( z ) = \frac{1}{K} .$$

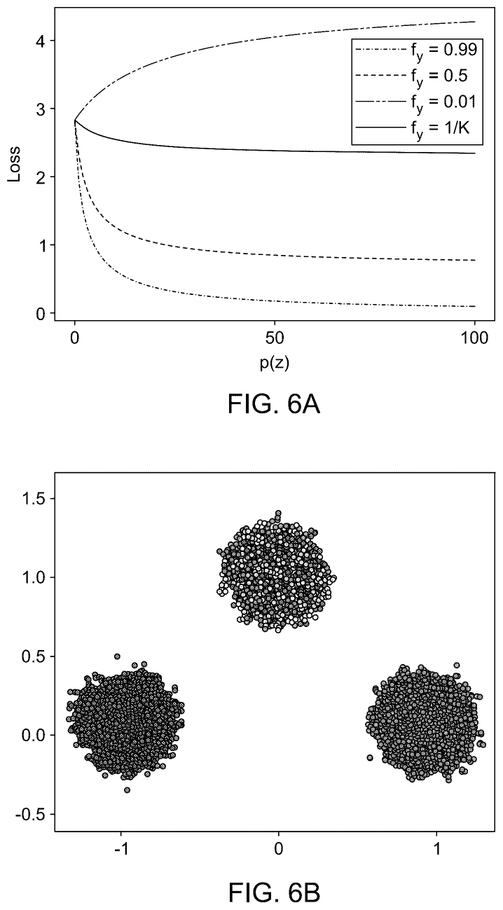

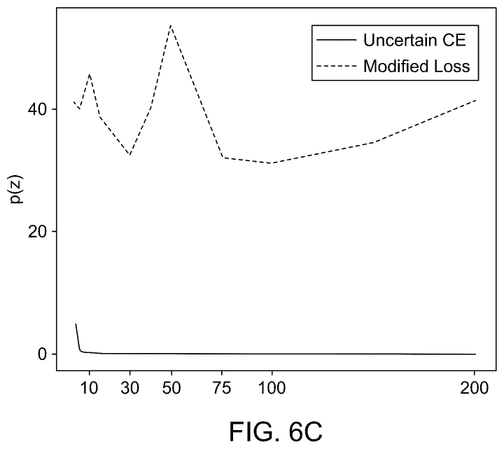

This implies that to minimize the loss function, we must decrease p(z) at these points, which seems counterintuitive since these z values correspond to objects from our training data. Although the issue concerning equation (5) arises primarily in the asymptotic context, it can readily be illustrated through the loss function profiles (see FIG. 6A) for a range of fixed correct class prediction probabilities.

To further emphasize the problem, the loss function landscape is examined and a demonstrative example is provided. Consider three two-dimensional Gaussian distributions, each with a standard deviation of 0.1 and means of −1, 0, and +1 with respect to the first coordinate, while the second coordinate of the mean is equal to 0 for all three clusters (see FIG. 6B). Left and right clusters are set to include objects of only one class, while the middle distribution contains uniformly distributed labels, representing a high aleatoric region. Each Gaussian contains an equal amount of data. The NatPN model is trained using a centralized approach, employing the loss function from equation (5). Subsequently, the quality of the learned model is evaluated by plotting the density in the center of middle Gaussian. Ideally, one would expect three peaks of equal size, and the density of the middle Gaussian stay constant with the number of classes K. However, it can be observed that the different picture with “density” estimate at the central cluster decreasing when one increases the number of clusters, see FIG. 6C.

It is essential to emphasize that addressing these issues is critical for the disclosed framework, there is a need to accurately differentiate between aleatoric and epistemic uncertainties in order to select the appropriate model for a given situation. Consequently, in the following section, it is proposed that a simple but efficient technique to rectify the aforementioned problem with the loss function, ensuring that our framework effectively distinguishes between the different types of uncertainties and makes informed model choices.

The solution still uses a parametric model to estimate density, but now a goal is to ensure that p(⋅) accurately represents the density of the training embeddings. To achieve this, the likelihood of the embeddings are explicitly maximized by incorporating a corresponding term into the loss function. Simultaneously, a goal is to prevent any potential impact of the Bayesian loss on the density estimation parameters, maintaining their independence.

Thus, the loss function is as follows:

$$L ( y , {StopGrad}_{p ( g ( x ) )} {α}^{post} ( x ) ) - λℋ [ Dir ( μ \vert {α}^{post} ( x ) ) ] - γ log p ( g ( x ) ) , \qquad ( 8 )$$

where λ, γ>0 are hyperparameters, and StopGrad_{p(g(x)} means that the gradient will be not propagated to the parameters of a density model, which parameterizes p(g(x)).

### A. Threshold Selection

Process to determine whether to make predictions using a local model or a global one.

This decision, resulting in a “switching” model, depends on a particular uncertainty score. This score can either be the logarithm of the density of embeddings, obtained using the density model (normalizing flows), or the entropy of predictive Dirichlet distribution (3). It is found that both measures provide comparable behavior, and in the experiments for the Table 2 density of embeddings is used, while for the Table 3 Dirichlet entropy is used.

To apply this approach, a rule is established for how a client decides whether to use its local model for predictions on a previously unseen input object x or to delegate the prediction to the global model. One approach is to select some uncertainty values' threshold. This threshold can be chosen based on an additional calibration dataset. In examples, each client's validation dataset is split in a 40/60 ratio, using the smaller part for calibration. Note that the calibration dataset only includes those classes used during the training procedure.

The choice of the threshold is arguably the most subjective part of the approach. Ideally, one would desire to have access to explicit out-of-distribution data (either from other client “local OOD” or completely unrelated data “global OOD”). With this data, uncertainty scores can be explicitly computed for both types of data (in-distribution and out-of-distribution) and the threshold can be selected that maximizes accuracy. However, it is unfair to have this data in the problem statement. Therefore, a procedure is to choose the threshold based solely on available local data.

To choose the threshold, it is assumed that for all clients, there might be a chance that some p % (typically 10%) of objects are outliers. The estimates of epistemic uncertainty (with either entropy or the logarithms of density of embeddings) are computed and used to select an appropriate threshold based on this assumption. For the high epistemic uncertainty points of the global model, a similar thresholding can be performed to optimize its prediction quality.

### Assessments

The effectiveness of the method is assessed through a series of thorough experiments. Seven diverse datasets are employed: MNIST, FashionMNIST, MedMNIST-A, MedMNIST-C, MedMNIST-S, CIFAR10, and SVHN. See Yann LeCun, Corinna Cortes, and Christopher Burges. The mnist database of handwritten digits. yann. lecun.com/exdb/mnist/, 1998; Han Xiao, Kashif Rasul, and Roland Vollgraf. Fashion-mnist: a novel image dataset for benchmarking machine learning algorithms. arXiv preprint arXiv:1708.07747, 2017; Jiancheng Yang, Rui Shi, and Bingbing Ni. Medmnist classification decathlon: A lightweight automl benchmark for medical image analysis. In IEEE 18th International Symposium on Biomedical Imaging (ISBI), pages 191-195, 2021; Jiancheng Yang, Rui Shi, Donglai Wei, Zequan Liu, Lin Zhao, Bilian Ke, Hanspeter Pfister, and Bingbing Ni. Medmnist v2-a large-scale lightweight benchmark for 2d and 3d biomedical image classification. Scientific Data, 10(1):41, 2023; A Krizhevsky. Learning multiple layers of features from tiny images. Master's thesis, University of Toronto, 2009; and Yuval Netzer, Tao Wang, Adam Coates, Alessandro Bissacco, Bo Wu, and Andrew Y Ng. Reading digits in natural images with unsupervised feature learning. 2011, each incorporated herein by reference in their entirety. The LeNet-5 encoder architecture is applied to the first five datasets, while ResNet-18 is used for CIFAR10 and SVHN. See Yann LeCun, Leon Bottou, Yoshua Bengio, and Patrick Haffner. Gradient-based learning applied to document recognition. Proceedings of the IEEE, 86(11):2278-2324, 1998; and Kaiming He, Xiangyu Zhang, Shaoqing Ren, and Jian Sun. Deep residual learning for image recognition. In Proceedings of the IEEE conference on computer vision and pattern recognition, pages 770-778, 2016, each incorporated herein by reference in their entirety.

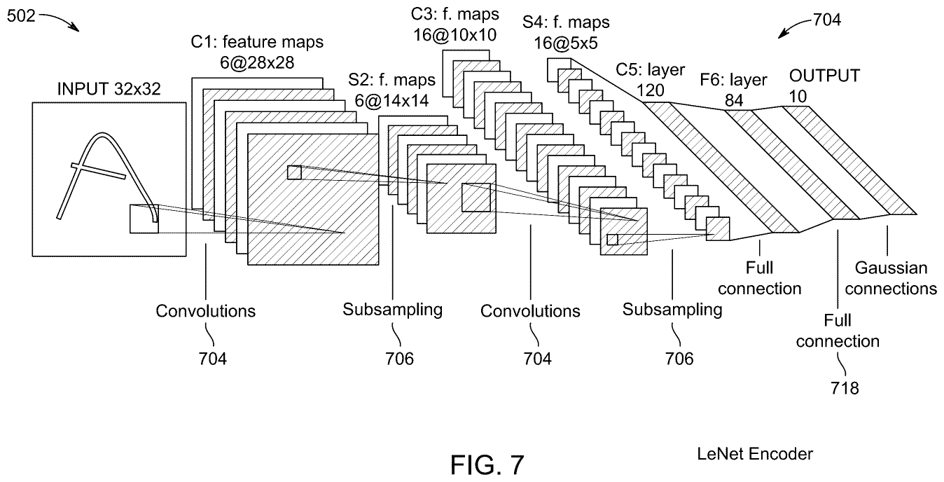

FIG. 7 is a flow diagram of an encoder for the network. In an embodiment, an encoder is the LeNet-5 CNN architecture which is made up of 7 layers. The layer composition consists of 3 convolutional layers 704, 2 subsampling layers 706 and 2 fully connected layers 718.

The Radial Flow normalizing flow is implemented due to its lightweight nature and inherent flexibility. See Danilo Rezende and Shakir Mohamed. Variational inference with normalizing flows. In International conference on machine learning, pages 1530-1538. PMLR, 2015, incorporated herein by reference in its entirety. In all examples, focus on a heterogeneous data distribution across clients. A scenario involves 20 clients, each possessing a random subset of 2 or 3 classes. However, the overall amount of data each client possesses is approximately equal. The federated learning process is conducted using FedAvg algorithm.

In the following sections, different experiments highlight the strengths of the approach. There is no dedicated experiment to illustrate how the approach deals with local ambiguous data as the vision datasets have very few points of this type.

### B. Assessing Performance of the Switching Model

In this section, the performance of method is assessed, where the prediction alternates between local and global models based on the uncertainty threshold.

For each dataset, three types of models are trained. First, a global model is trained as a result of the federated procedure, following FedAvg procedure. Then, every client, after the federated procedure, retains the resulting encoder network while the classifier and flow are retrained from scratch using only local data. The third type of model, the “switching” model, alternates between the first two models based on the uncertainty threshold set for each client.

Additionally, for each client, the following three types of data are considered: data of the same classes used during training (InD), data of all other classes (OOD), and data from all classes (Mix). For each of these models, datasets, and data splits, the average prediction accuracy (client-wise) is computed. The results of this experiment are presented in Table 2.

TABLE 2
The table presents the average (per-client) predictive accuracy of various models, segmented
according to the client's training distribution. The data is partitioned into three sets:
InD (comprising only the classes seen during training). OOD (all other classes), and Mix
(a combination of both, representing the entire data domain). It is evident from the table
that alternating between the local and global models based on the model confidence helps
enhance the predictive performance of both models in the most realistic scenario represented
by the Mix set (third column). For the Mix set the best performing method is in bold.

| | InD | | | OOD | | | Mix | | |
|---|---|---|---|---|---|---|---|---|---|
| Dataset | Local | Global | Switch | Local | Global | Switch | Local | Global | Switch |
| MNIST | 99.7 | 98.0 | 98.4 | 0.0 | 98.1 | 97.7 | 49.8 | 98.0 | 98.0 |
| FashionMNIST | 97.7 | 63.6 | 85.5 | 0.0 | 69.1 | 61.8 | 48.9 | 66.3 | 73.7 |
| MedMNIST-A | 97.1 | 65.5 | 83.6 | 0.9 | 67.2 | 59.8 | 49.3 | 63.9 | 69.5 |
| MedMNIST-C | 81.6 | 55.2 | 71.9 | 2.2 | 59.8 | 34.1 | 41.8 | 54.6 | 53.2 |
| MedMNIST-S | 85.1 | 43.2 | 64.6 | 0.1 | 50.2 | 29.5 | 42.7 | 39.7 | 43.7 |
| CIFAR10 | 90.0 | 30.8 | 67.6 | 0.1 | 29.5 | 26.9 | 45.0 | 30.1 | 47.2 |
| SVHN | 95.9 | 36.0 | 84.5 | 0.9 | 32.0 | 29.2 | 48.6 | 37.2 | 59.8 |

From the Table 2, it can be observed that the “switching” model's performance for InD data is always better than the one for the global model though inferior to the local one. For the OOD data, it is evident that the “switching” model improves over the local model's performance. This situation corresponds to the “local OOD (unknown knowns)” case from Table 1, illustrating the benefits of referring to global knowledge when the local model is unfamiliar with the data. The third column illustrates a scenario where the input data includes whole domain of the training data. This is the most realistic practical scenario—all the clients aim to collaboratively solve the same problem, given different local data. However, due to the heterogeneous nature of the between-clients data distribution (covariate shift), local models cannot learn the entire data manifold. Therefore, occasionally referring to global knowledge is beneficial while still preserving personalization when the local model is confident. We observe that the “switching” model outperforms both local and global models on 6 datasets out of 8 while showing the same performance with the global model on MNIST.

### C. Assessment in the Presence of Global Out-of-Distribution (Unknown-Unknown) Data

In this section, the method's behavior is assessed in the presence of both—“soft-OOD” (unknown knowns or local OOD) and “hard OOD” (unknown unknowns or global OOD).

The following is the experimental setup. Subsequently, a pair of datasets are identified—the first is the entire InD dataset, fragments of which were distributed among federated clients during training. The second dataset represents explicit OOD data, unassociated with the federated training procedure. A primary objective is to evaluate the performance of models when faced with various types of data. The following scheme achieves this.

Firstly, both datasets are fed into the local model which, based on the predetermined threshold, labels each input as either in-distribution or out-of-distribution. Subsequently, the local model makes prediction for the objects classified as in-distribution by it, and the remaining objects are further processed by the global model.

The process of selecting a threshold for the global model is arguably even more complex than for the local model. In practice it might be determined based either on a local data of each particular client or possibly based on the separate global calibration data. In experiments this threshold is varied in order to evaluate how the model's performance metric depend on the fraction of filtered objects. A quality measure includes the precision of predictions of the model for a given client. Apparently, if OOD objects are filtered well, then the precision improves with the fraction of filtered objects as less false positives are obtained.

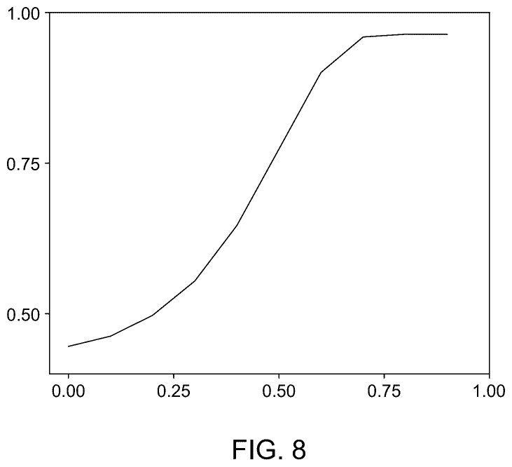

First, FIG. 8 illustrates how for the task with InD MNIST dataset and OOD FashionMNIST precision improves with increased fraction of the filtered data. It shows that the considered uncertainty metric filters well uncertain objects thus improving the performance. Then, in Table 3, the average increase in precision is provided for when 50% for the considered datasets. There are 50% of OOD samples in the data and, thus, in the ideal case filter out all the OOD data for the selected threshold. It can be clearly observed that the significant increase in precision over all the datasets. Some additional experimental results for this experiment can be found in SM.

TABLE 3
The table shows improvement (in percentage) of precision,
given that 50% of the objects were filtered by their
uncertainty, evaluated using global model.

| In- | Out-of- | Prec. |
|---|---|---|
| distribution | distribution | impr. |
| MNIST | FashionMNIST | 42.01 |
| FashionMNIST | MNIST | 28.34 |
| CIFAR10 | SVHN | 27.94 |
| SVHN | CIFAR10 | 41.52 |
| MedMNIST-A | FashionMNIST | 26.88 |
| MedMNIST-S | FashionMNIST | 15.57 |
| MedMNIST-C | FashionMNIST | 11.60 |

A personalized federated learning framework that leverages both a globally trained federated model and personalized local models to make final predictions is described. The selection between these models is based on the confidence in the prediction.

An empirical evaluation demonstrated that, under realistic scenario, this approach outperforms both local and global models when used independently. While the model's capacity to handle out-of-distribution data is not perfect and depends on various factors, such as the quality of the global model and the selection of the threshold, the “switching” approach ultimately leads to improved performance. It also enhances the reliability of AI applications, underscoring the methodology in a broader context of federated learning environments.

As mentioned above, the model consists of encoder, parametrized by ϕ, density model (normalizing flow in our case), parametrized by ψ, and classifier model with parameters θ. All these parameters were learned in federated fashion, and effectively “felt” all the data, distributed over clients. In contrast, when training local models (train only flow and classifier from scratch, encoder is fixed to the one after federated training), use only local data.

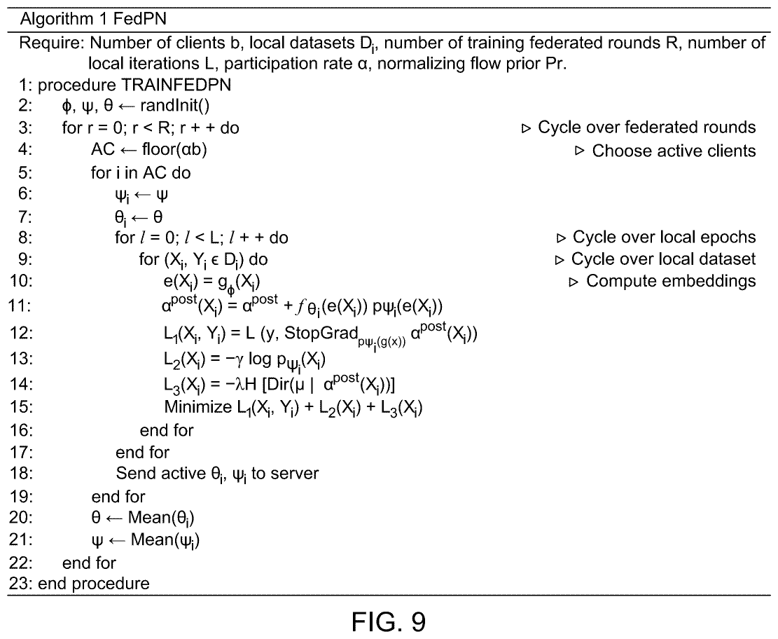

To train the global normalizing flow in a federated fashion, use the FedAvg naive approach, which begins with the same initialization each round, and then trains each flow locally. During inference on an unseen data object x at client i, differentiate between θ, ψ and θᵢ, ψᵢ based on some uncertainty scores, computed using the density of g_{ϕ}(x) (density of embedding). See Algorithm 1 in FIG. 9 for the detailed procedure.

### Loss Function

In an asymptotic form of (5), for all x>0, the following inequality holds:

$$log x - 1 / x \leq ψ ( x ) \leq log x - 1 / 2 x$$

Recalling the update rule (1) and using the specific parameterization of

$${a}_{c}^{prior} = 1$$

for all c,

$${a}_{c}^{post} > 1 .$$

Hence, approximate

$$ψ ( {a}_{c}^{post} ) \approx log ( {a}_{c}^{post} ) .$$

To simplify the notation, use z=g(x):

$$\begin{matrix}L ( y , {α}_{c}^{post} ) ) \approx ( {α}_{0}^{post} ( x ) ) - log ( {α}_{y}^{post} ( x ) ) = log [ \frac{{\sum }_{i = 1}^{K} {α}_{i}^{prior} + p ( z ) {\sum }_{i = 1} {f}_{i} ( z )}{{α}_{y}^{prior} + p ( z ) {f}_{y} ( z )} ] \\ = log {\sum }_{i = 1}^{K} {α}_{i}^{prior} + log [ \frac{1 + p ( z ) \frac{1}{{\sum }_{i = 1}^{K} {α}_{i}^{prior}}}{{α}_{y}^{prior} + p ( z ) {f}_{y} ( z )} + 1 - 1 ] \\ = log {\sum }_{i = 1}^{K} {α}_{i}^{prior} + log [ 1 + \frac{1 - {α}_{y}^{prior} + p ( z ) ( \frac{1}{{\sum }_{i = 1}^{K} {α}_{i}^{prior}} - {f}_{y} ( z ) )}{{α}_{y}^{prior} + p ( z ) {f}_{y} ( z )} ] \\ = log {\sum }_{i = 1}^{K} {α}_{i}^{prior} + log [ 1 + \frac{1 + {α}_{y}^{prior}}{{α}_{y}^{prior} + p ( z ) {f}_{y} ( z )} + \frac{p ( z ) ( \frac{1}{{\sum }_{i = 1}^{K} {α}_{i}^{prior}} - {f}_{y} ( z ) )}{{α}_{y}^{prior} + p ( z ) {f}_{y} ( z )} ]\end{matrix} , \qquad ( 9 )$$

where

$$\sum \frac{K}{i = 1} {f}_{i} ( z ) = 1$$

is a softmax vector,

$${a}_{c}^{prior} = 1 .$$

This cancels the second term under the logarithm. Note that this term motivates p(z) to concentrate mass on training examples, irrespective of the probability of the correct class f_{y}(z).

### Parameterization of Normalization Flow

In this subsection, the specifics of incorporating the normalizing flow into the pipeline are described. A critical advantage of the approach, in comparison with the one presented in, lies in the utilization of a single normalizing flow as opposed to learning individual flows for each class. This strategy proves to be highly effective particularly when the dimensionality of embeddings remains relatively low.

Nevertheless, there is an increased complexity in the single flow's ability to approximate multiple modes corresponding to distinct classes in the context of higher-dimensional spaces. To address this problem, train of one flow per class. However, the same parameterization is preserved, effectively marginalizing the class labels. Subsequently, the density of the encoder network's embeddings is computed as follows:

$$p ( z ) = {{\sum }_{c = 1}}^{K} p ( z , c ) = {{\sum }_{c = 1}}^{K} p ( z \vert c ) p ( c ) ,$$

where z represents the embedding (output of the encoder network), p(z|c) is the density of z with respect to the normalizing flow with index c, and p(c) denotes the prior probability of class c.

The prior class probabilities are estimated based on each client's data, attaching the relative proportion of the class. If a particular class is not represented in a client's data, the corresponding class probability is set to zero, thus the flow's parameters are not updated. It is worth noting that as the global model has no access to the clients' data, a uniform distribution is set over the classes.

### Hyperparameters Selection

In this section, the selection process is described for the hyperparameters associated with the framework. To ease the reading experience, consolidate all the hyperparameters into a tabulated format. For the summary, see Table 4.

When the federated training process is concluded, the resulting encoder is kept for all the client models as fixed, and reinitialize the flow and classifier models. Subsequently, only the flow and the classifier models are trained, using only the data specific to a given client. This training phase lasts for 10 local epochs using Adam optimizer with learning rate of 1e−3 and the same y as in the federated stage.

TABLE 4
Summary of the hyperparameters, used for the training of the framework.

| | Batch | | Learning | | |
|---|---|---|---|---|---|
| Dataset | size | Optimizer | rate | Momentum | Architecture |
| MNIST | 64 | SGD | 0.01 | 0.9 | LeNet-5 |
| FashionMNIST | 64 | SGD | 0.01 | 0.9 | LeNet-5 |
| MedMNIST-A | 64 | SGD | 0.01 | 0.9 | LeNet-5 |
| MedMNIST-C | 64 | SGD | 0.01 | 0.9 | LeNet-5 |
| MedMNIST-S | 64 | SGD | 0.01 | 0.9 | LeNet-5 |
| CIFAR10 | 64 | SGD | 0.01 | 0.9 | ResNet-18 |
| SVHN | 64 | SGD | 0.01 | 0.9 | ResNet-18 |

| | Logprob | Entropy | Federated | Local |
|---|---|---|---|---|
| Dataset | weight γ | weight λ | rounds | epochs |
| MNIST | 0.001 | 0.0 | 100 | 10 batches |
| FashionMNIST | 0.001 | 0.0 | 100 | 10 batches |
| MedMNIST-A | 0.001 | 0.0 | 100 | 10 batches |
| MedMNIST-C | 0.001 | 0.0 | 100 | 10 batches |
| MedMNIST-S | 0.001 | 0.0 | 100 | 10 batches |
| CIFAR10 | 0.001 | 0.0 | 100 | 10 batches |
| SVHN | 0.001 | 0.0 | 100 | 10 batches |

### Additional Examples

### Distinction Between Aleatoric and Epistemic Uncertainties

In the above only two parts of the framework are provided, resulting in a “switching” model, where one could abstain from the prediction of the local model and use the global model instead, or to abstain from the prediction at all (if the certainty of global model is low).

In another part of the pipeline, the ability to abstain from the prediction if aleatoric uncertainty (label noise or input ambiguity) is sufficiently big. Typical benchmark datasets like the ones used are carefully curated and are found to have almost no label noise. Consequently, illustrating aleatoric uncertainty based solely on the information derived from these datasets can pose significant challenges.

Nevertheless, to facilitate the comprehensive execution of the pipeline, artificial label noise is introduced. This strategic introduction serves a twofold purpose—not only does it effectively demonstrate the model's capability to discern between aleatoric and epistemic uncertainties, but it also showcases that the model is unlikely to transition to a global model in the presence of high aleatoric uncertainty.

To conduct this examination, the labels of five classes are changed—specifically, classes 5, 6, 7, 8, and 9—within the MNIST dataset. The remainder of the classes, namely classes 0, 1, 2, 3, and 4, are retained in their original, unaltered state, thereby maintaining a degree of purity and control within the experiment.

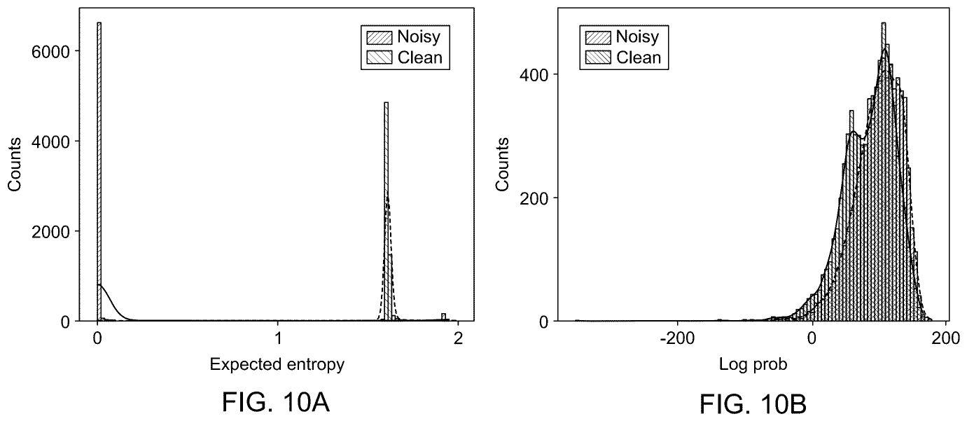

The results of this experiment are presented in FIGS. 10A, 10B. In FIG. 10A, the histogram depicts aleatoric uncertainty, represented as the expected entropy of the predictive distribution. It was computed using the formula specified in equation (4). In FIG. 10B, the histogram demonstrates epistemic uncertainty. It is computed using the logarithm of the density of the embeddings. It can be seen that the aleatoric uncertainty, evaluated as an expected entropy of predictive distribution, drastically differs for noisy labels and for clean ones. Apparently, the threshold for the separation can be easily chosen using this histogram (e.g., the value of 1). Contrary to the measure of aleatoric uncertainty, the scores of epistemic uncertainty (here it is logarithm of the density of features) cannot distinguish between noisy and clean labels.

The above-described framework effectively allows to distinguish between noisy labels through the disentanglement of uncertainties into aleatoric and epistemic parts. This allows to abstain from the prediction (when the label noise), or delegate inference to the global model (when input is out-of-distribution).

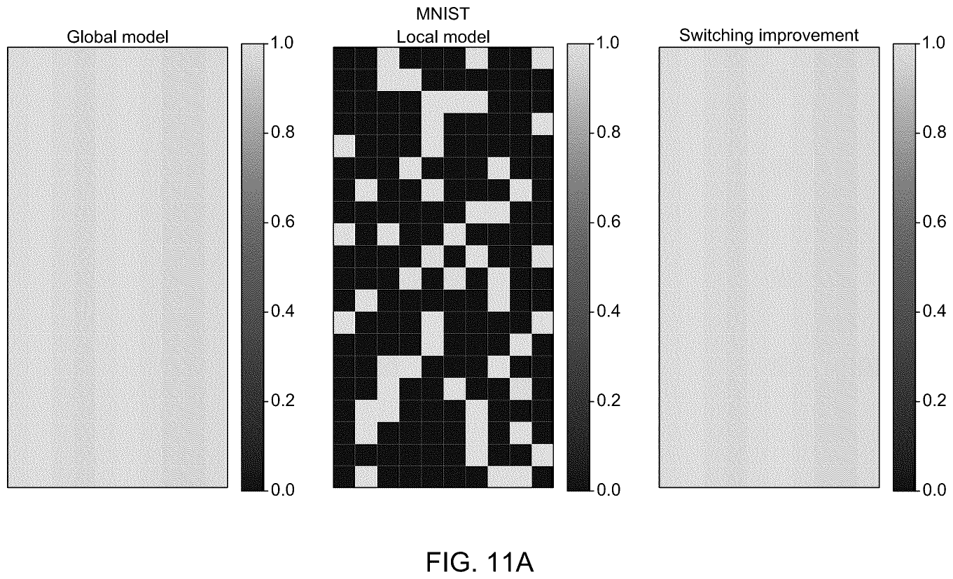

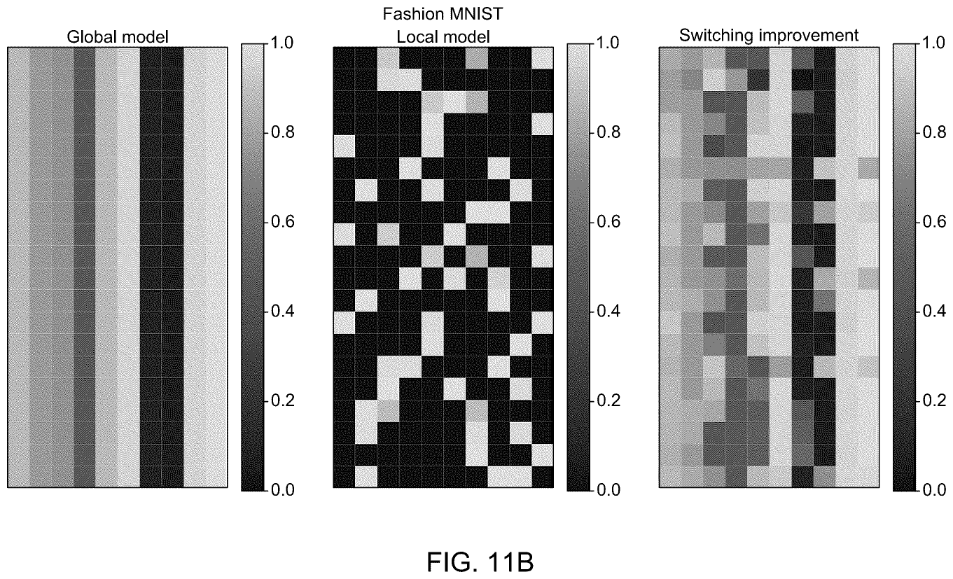

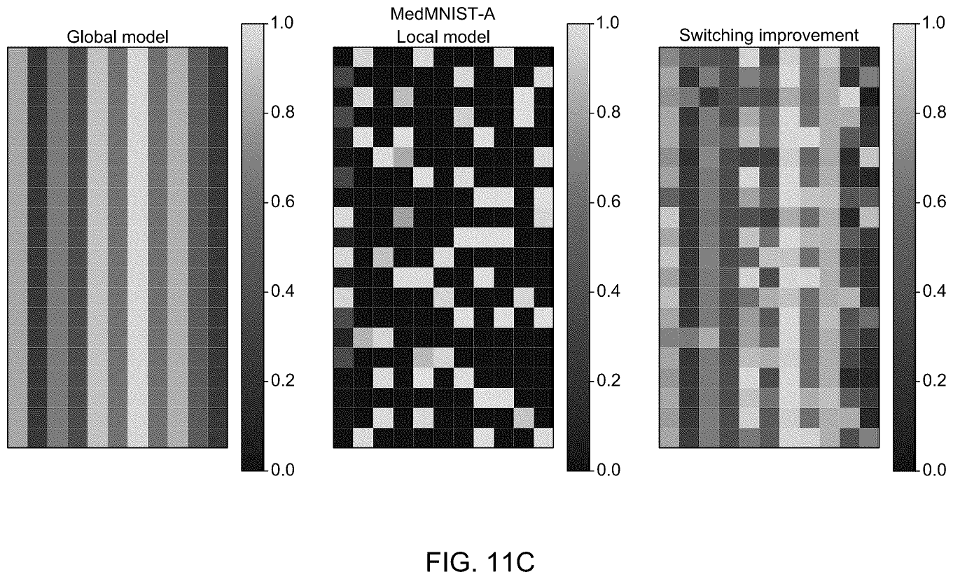

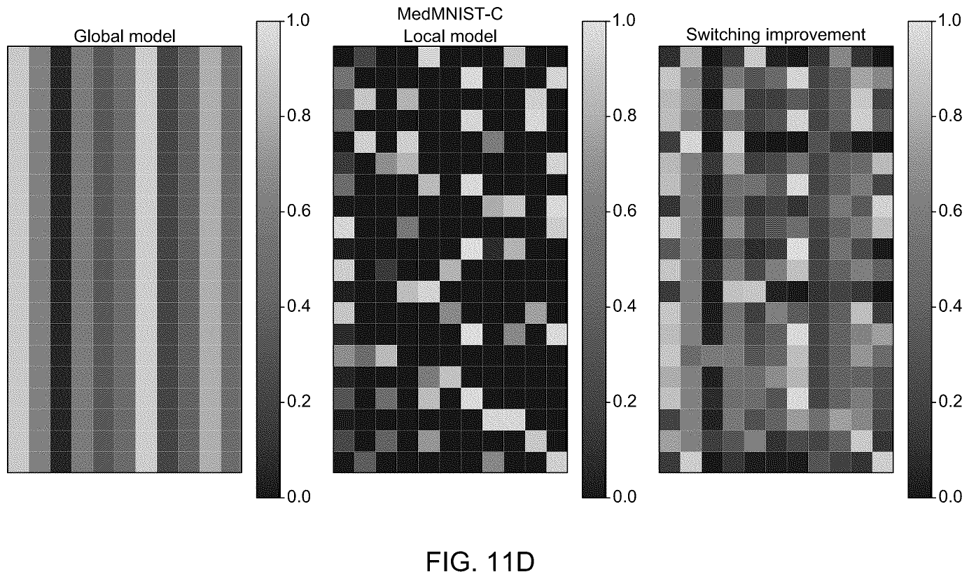

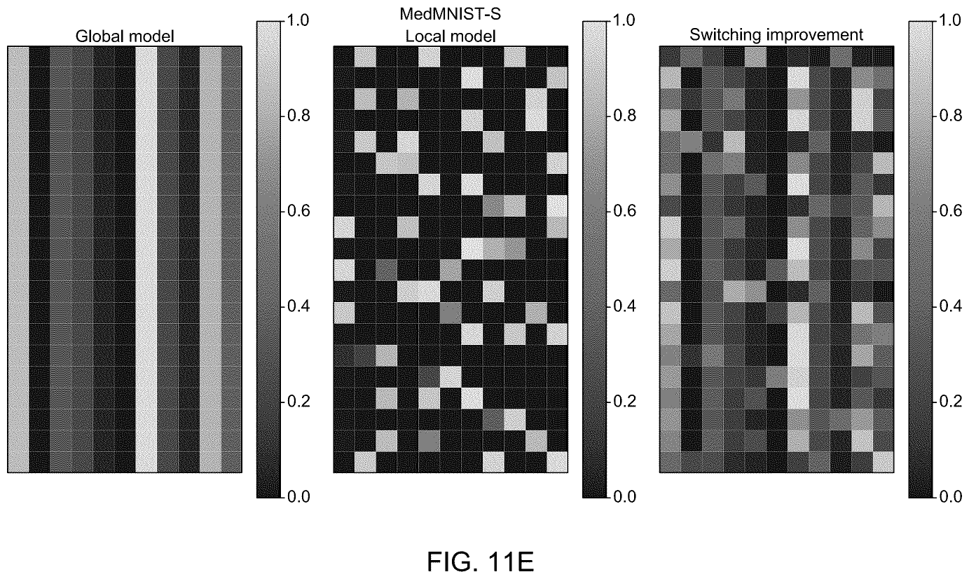

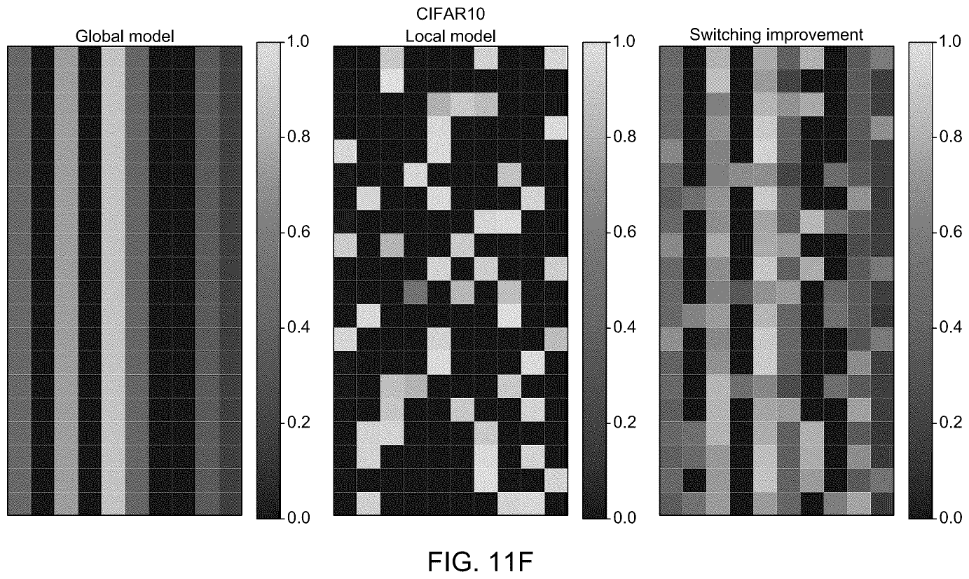

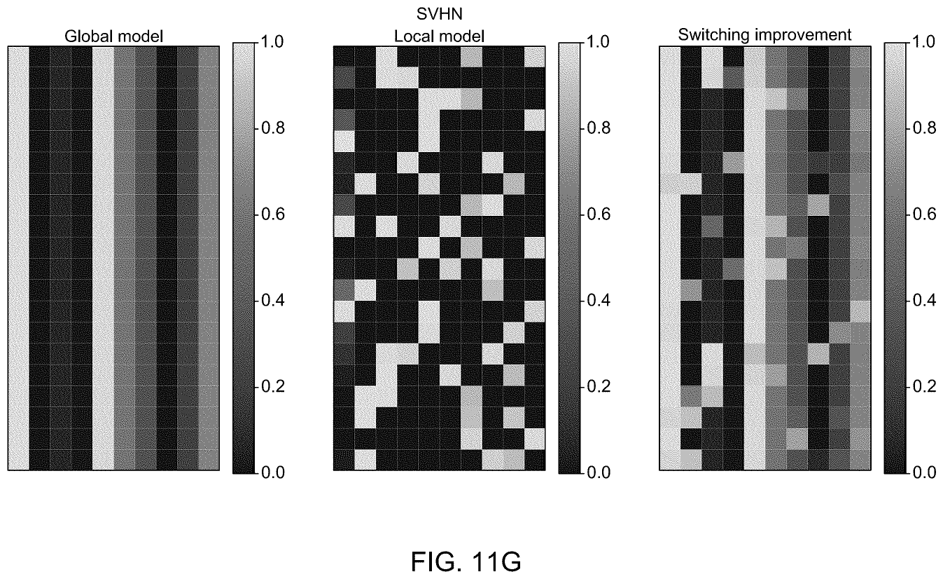

This section provides additional insights supplementing the experimental results presented above and summarized in Table 2. Specifically, the variations in accuracy of learned models are explored on a per-class basis. Results are presented in FIGS. 11A-11G.

FIGS. 11A-11G displays matrices of accuracy for varying models, each assessed across distinct classes. Rows: Each row is associated with a specific client, as identified by its index. Columns: Each column represents a unique class number. Moving from left to right, each matrix depicts the accuracy of the corresponding model (global, local, or “switching”) as evaluated on a particular class.

For each dataset under consideration, a trio of heatmaps are presented. Every heatmap comprises 20 rows, where each row signifying a client, and a number of columns, where each column representing a specific class. The elements of these heatmaps, found at the intersection of the i-th row and j-th column, contain the accuracy achieved by the model of the i-th client and evaluated using the j-th class.

The leftmost heatmap represents the global model performance, which is identical for each client, hence exhibiting a striped structure. The central heatmap corresponds to the local models, exhibiting a distinctive structure-accuracy of classes featured in the training dataset approaches 1, whereas that of unrepresented classes are almost 0, as anticipated.

The rightmost heatmap shows the resulting per-class accuracy of switching model, underscoring the model's improvement on classes absent from the clients' training datasets.

The rightmost heatmap also reveals a degradation in accuracy for classes that were present in the local datasets. This discrepancy may be because the feature representations learned by the encoder are not perfectly differentiated for the classes, resulting in some degree of overlap.

In this context, a balance in setting the threshold for the switching operation becomes challenging. In traditional centralized scenarios, techniques such as spectral normalization are commonly employed to mitigate feature collapse and enhance the distinctiveness of these features.

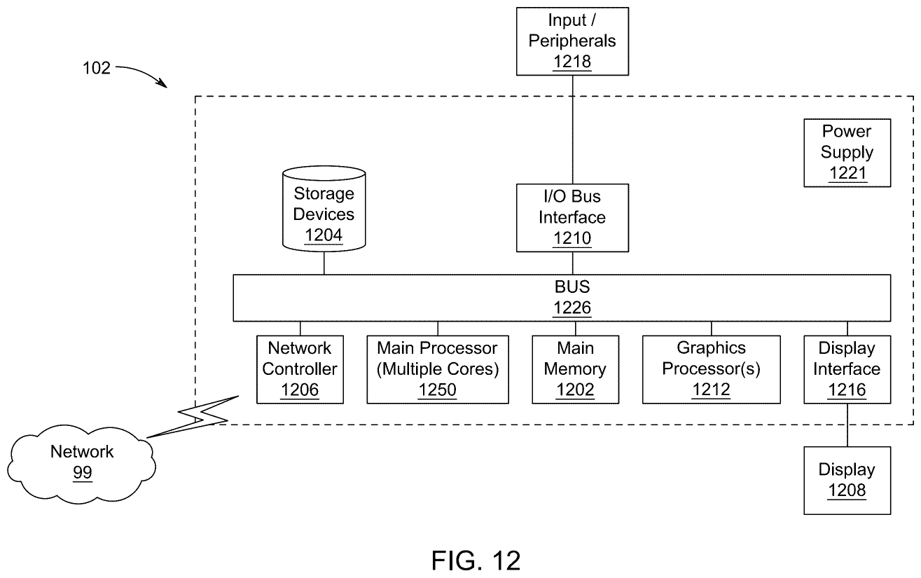

FIG. 12 is a block diagram illustrating an example computer system for implementing the machine learning training and inference methods according to an exemplary aspect of the disclosure. The computer system may be an AI workstation running an operating system, for example Ubuntu Linux OS, Windows, a version of Unix OS, or Mac OS. The computer system 1200 may include one or more central processing units (CPU) 1250 having multiple cores. The computer system 1200 may include a graphics board 1212 having multiple GPUs, each GPU having GPU memory. The graphics board 1212 may perform many of the mathematical operations of the disclosed machine learning methods. The computer system 1200 includes main memory 1202, typically random access memory RAM, which contains the software being executed by the processing cores 1250 and GPUs 1212, as well as a non-volatile storage device 1204 for storing data and the software programs. Several interfaces for interacting with the computer system 1200 may be provided, including an I/O Bus Interface 1210, Input/Peripherals 1218 such as a keyboard, touch pad, mouse, Display Adapter 1216 and one or more Displays 1208, and a Network Controller 1206 to enable wired or wireless communication through a network 99. The interfaces, memory and processors may communicate over the system bus 1226. The computer system 1200 includes a power supply 1221, which may be a redundant power supply.

In some embodiments, the computer system 1200 may include a server CPU and a graphics card by NVIDIA, in which the GPUs have multiple CUDA cores. In some embodiments, the computer system 1200 may include a machine learning engine 1212.

The above-described hardware description is a non-limiting example of corresponding structure for performing the functionality described herein.

Numerous modifications and variations of the present disclosure are possible in light of the above teachings. It is therefore to be understood that the invention may be practiced otherwise than as specifically described herein.

1. A medical diagnosis system in a network, comprising:
a plurality of workstations for a plurality of respective medical facilities, where each workstation performs medical diagnosis using medical data that is unique to the respective medical facility;
a central server, connected to communicate with the plurality of workstations, for maintaining a global model for the medical diagnosis;
wherein each of the workstations maintains a local model for the medical diagnosis,
wherein each of the workstations includes a selector configured to switch between:
(i) use of the global model for the medical diagnosis only if the local model has high epistemic uncertainty about the diagnosis at a given input point, wherein the local model has high epistemic uncertainty above a predetermined uncertainty threshold that is based on a quantity of data about a particular input point that is less than a predetermined quantity,
(ii) use of the local model for the medical diagnosis when the local model is confident, either in predicting a particular medical diagnosis or when predicting an ambiguous diagnosis with aleatoric uncertainty that is above the predetermined uncertainty threshold.

2. The medical diagnosis system of claim 1, wherein the local models of each of the workstations and the global model each determine an aleatoric uncertainty for the medical diagnosis,
wherein when the aleatoric uncertainty is above the predetermined uncertainty threshold, both the local model and the global model abstain from prediction.

3. The medical diagnosis system of claim 1, wherein each of the workstations is configured to determine the predetermined uncertainty threshold based on a calibration dataset.

4. The medical diagnosis system of claim 1, wherein the local model is trained with local hospital patient data,
wherein when the local hospital patient data does not fit a local data statistical distribution, the selector of a respective workstation switches to downloading trained global weights of the global model to the local model.

5. The medical diagnosis system of claim 1, wherein the local models each are a neural network trained as a Dirichlet model, including a normalization flow and a decoder mapping extracted features to a vector of class probabilities, wherein an encoder performs the feature extraction function that maps an input to a lower-dimensional embedding.

6. The medical diagnosis system of claim 5, wherein each of the workstations comprises a training layer that performs training of the local models using a training loss function that simultaneously maximizes likelihood of training embeddings and prevents an impact of a uncertain cross entropy loss on density estimation parameters of the Dirichlet model.

7. The medical diagnosis system of claim 6, wherein the prevention by the training loss function is preventing propagation of a training gradient to the density estimation parameters of a parametric model to estimate density.

8. The medical diagnosis system of claim 1, wherein the global model includes an encoder, having encoder parameters, a density model, having density parameters, and a classifier model with classifier parameters, wherein the global model is trained as an average over parameters of the local models.

9. The medical diagnosis system of claim 8, wherein the local models each include includes an encoder, having encoder parameters, a density model, having density parameters, and a classifier model with classifier parameters, wherein, after federated learning, the local models are trained through training the density model and the classifier model using local medical data, while keeping the encoder parameters fixed with global encoder parameter values.

10. The medical diagnosis system of claim 1, wherein the predetermined uncertainty threshold is quantile [0.8, 0.9].

11. A method of medical diagnosis in a network including a plurality of workstations for a plurality of respective medical facilities, where each workstation maintains a local model for medical diagnosis, and a central server, connected to communicate with the plurality of workstations, for maintaining a global model for the medical diagnosis, the method comprising:
performing, in the plurality of workstations, the medical diagnosis using medical data that is unique to the respective medical facility;
switching, in each of the workstations, between
(i) use of the global model for the medical diagnosis only if the local model has high epistemic uncertainty about the diagnosis at a given input point, wherein the local model has high epistemic uncertainty above a predetermined uncertainty threshold that is based on a quantity of data about a particular input point that is less than a predetermined quantity,
(ii) use of the local model for the medical diagnosis when the local model is confident, either in predicting a particular medical diagnosis or when predicting an ambiguous diagnosis with aleatoric uncertainty that is above the predetermined uncertainty threshold.

12. The method of claim 11, further comprising determining, by each of the local models and the global model, an aleatoric uncertainty for the medical diagnosis,
wherein when the aleatoric uncertainty determined by one local model is above the predetermined uncertainty threshold, both the one local model and the global model abstain from prediction.

13. The method of claim 11, further comprising determining, by each of the workstations, the predetermined uncertainty threshold based on a calibration dataset.

14. The method of claim 11, wherein the local model is trained with local hospital patient data,
the method further comprising
when the local hospital patient data does not fit a local data statistical distribution, switching, by a respective workstation, to downloading trained global weights of the global model to the local model.

15. The method of claim 11, wherein the local models each are a neural network trained as a Dirichlet model, including a normalization flow and a decoder mapping extracted features to a vector of class probabilities, further comprising performing a feature extraction function to map, by an encoder, an input to a lower-dimensional embedding.

16. The method of claim 15, further comprising training, by each of the workstations, the local models using a training loss function that simultaneously maximizes likelihood of training embeddings and prevents an impact of a uncertain cross entropy loss on density estimation parameters of the Dirichlet model.

17. The method of claim 16, wherein the prevention by the training loss function is preventing propagation of a training gradient to the density estimation parameters of a parametric model to estimate density.

18. The method of claim 11, wherein the global model includes an encoder, having encoder parameters, a density model, having density parameters, and a classifier model with classifier parameters, the method further comprising training the global model as an average over parameters of the local models.

19. The method of claim 18, wherein the local models each include includes an encoder, having encoder parameters, a density model, having density parameters, and a classifier model with classifier parameters, the method further comprising, after federated learning, training the local models through training the density model and the classifier model using local medical data, while keeping the encoder parameters fixed with global encoder parameter values.

20. The method of claim 11, wherein the predetermined uncertainty threshold is quantile [0.8, 0.9].
